(19) United States
(12) Patent Application Publication
(10) Pub. No.: US 2026/0159873 A1
(43) Pub. Date: Jun. 11, 2026

(54) Method and Apparatus for the Purifying Nucleic Acids, in particular for Nucleic Acid Amplification

(71) Applicant: Robert Bosch GmbH, Stuttgart (DE)

(72) Inventor: Bernd Faltin, Gerlingen (DE)

(21) Appl. No.: 19/179,641

(22) Filed: Apr. 15, 2025

(30) Foreign Application Priority Data
Apr. 19, 2024 (DE) 10 2024 203 643.9

(51) Int. Cl. C12Q 1/6806; B01L 3/00

(52) U.S. Cl. CPC C12Q 1/6806; B01L 3/502761; B01L 2200/0647; B01L 2300/0681

(57) ABSTRACT

A method for purifying nucleic acids from cells in a sample with a microfluidic apparatus is disclosed. Further, an microfluidic apparatus configured for this purpose is also disclosed.

This application claims priority under 35 U.S.C. § 119 to application no. DE 10 2024 203 643.9, filed on Apr. 19, 2024 in Germany, the disclosure of which is incorporated herein by reference in its entirety.

### BACKGROUND

For the detection of pathogens, nucleic acids can be isolated from a sample containing biological cells, referred to as nucleic acid extraction, to be subsequently selectively identified as a pathogen, in particular via nucleic acid amplification as in PCR. The use of such methods at the point of care and as patient-facing instant diagnostics can be implemented with microfluidic systems, wherein a patient sample is entered into a microfluidic cartridge and the cartridge is actuated in an analytical device for performing nucleic acid amplification inside the cartridge.

In the course of nucleic acid extraction to prepare the nucleic acid amplification, the so-called Bind-Wash-Elute method (BWE) firstly decomposes the cells in the sample to be tested in a suitable manner, i.e. chemically, thermally, or mechanically lysed, and also releases the nucleic acids from bacteria, viruses, or fungi in the sample. Because the samples are typically introduced directly into a liquid transport medium after receipt and mixed with the transport medium into the microfluidic system, the transport media used for this purpose, such as in particular Copan eNAT™, can support the lysis depending on the chemical composition and can stabilize the released nucleic acids during transport and storage. Thus, the nucleic acids already partially released are mixed with the residues of the sample and the transport medium together and input into the cartridge. For a possibly more thorough lysis, an additional lysing buffer can be added to the sample in the cartridge.

Typically, the lysed sample in the cartridge is mixed with a binding buffer and, in a first step of the BWE method, is brought into contact with a solid phase, in particular a silica filter, of the cartridge, wherein the nucleic acid is adsorbed and retained by interaction at the surface of the solid phase, while the residues of the mixture of sample, transport medium, and binding buffer as well as any possible lysing buffer is removed from the solid phase, in particular through the filter. This separation of the nucleic acids from other constituents, in particular via a solid phase, is commonly referred to as purification. In suitable cases, the lysing buffer can be used at least as part of the binding buffer, because the chaotropic substances typically contained in the lysing buffer support the binding of the nucleic acid to the solid phase, in particular to a silica filter. Following the binding, any remaining disruptive residues, for example, cell debris, are removed from the solid phase by a suitable wash buffer, which will also be considered part of the purification. By selecting a suitable elution buffer, the nucleic acids are dissolved under suitable conditions, in particular at an elevated temperature from the solid phase, and can be available for the downstream amplification reactions, for example for a polymerase chain reaction or an isothermal amplification method commonly referred to as nucleic acid amplification techniques (NAAT).

In the BWE method, the choice of the lysing and binding medium is essential, because these media come into contact with the usually liquid sample, are in particular mixed together, and thus the chemical conditions of the environment of the released nucleic acids are co-determined, which are decisive for the success of binding the nucleic acids to the solid phase. For the suitable chemical conditions for selectively purifying the nucleic acids at the solid phase, chaotropic salts or alcohol-containing solutions, for example ethanol, in particular, can be used, which, however, can inhibit or completely disrupt subsequent nucleic acid amplification. It is therefore essential that residues of the lysing reagents and the binding buffer, in particular the chaotropic substances, are also removed by washing the nucleic acids bound on the solid phase.

However, if the patient sample containing the biological cells is received in a non-lysing transport medium, in particular a transport medium without lysing properties, such as UTM® by Copan or Amies medium, in order to keep the cells intact, on the other hand, the sample is initially prepared after input and before release of the nucleic acids via a suitable solid phase, in particular a filter, of the cartridge, in order to retain these cells via size exclusion, in particular based on a chosen pore size of the filter, and separate them from the transport medium and other sample constituents, which is also called the cell capture approach. A wash buffer positioned upstream on the cartridge can also be used, which separates remaining sample residues from the retained cells and dissipates them from the solid phase. The cells are then lysed with a lysing medium, which releases the nucleic acids contained in the cells and thereby also leads them away from the solid phase, or in the case of a pore filter, through the pores of the filter. The lysing medium with the released nucleic acids contained therein is then mixed with reagents for performing the nucleic acid amplification, wherein these reagents, in particular a so-called master mix, for example in the form of a bead, are also usually positioned upstream in the cartridge. Because the lysing medium is directly incorporated into the reaction mixture, the lysing medium used must not contain substances that interfere with nucleic acid amplification. Thus, a lysing medium typically used for the BWE method with chaotropic substances, which is also referred to as a hard lysing medium, is generally not considered for the cell capture method, and a detergent such as octoxinol 9 (Triton®-X-100) is used as a non-harmful for nucleic acid amplification and also described as soft lysing medium, as described for example in the publication DE 10 2020 203 035 A1.

### SUMMARY

In light of this, the disclosure relates to a method for purifying nucleic acids from cells in a sample with a microfluidic apparatus, for example as preparation for a nucleic acid amplification, for example for duplication and detection of nucleic acid sequences of pathogens in the sample.

A sample can be in particular a biological sample, i.e. a sample comprising a portion of a living being, in particular a body fluid or a portion of a body tissue, for example blood, sputum, urine, or a swab. Biological cells are understood here to mean cells of multi-celled living organisms such as mammals, including humans or single-celled organisms such as bacteria, but also viruses or fungi. After receipt from a living being, in particular a patient, the sample is in particular received into a medium for transport from the living organism to the analysis, i.e. up to the microfluidic apparatus, which is referred to as transport medium and is preferably suitable for the maintenance of at least certain biological cells contained in the sample. The mixture comprising the biological sample and such a transport medium is thus also referred to as a sample. A transport medium for the maintenance of at least certain biological cells contained in the sample, hereinafter also referred to as a stabilizing transport medium, is in particular a liquid transport medium for the maintenance of microorganisms, in particular pathogens in the form of bacteria, viruses, fungi, or other individual single-celled organisms. For example, the medium is, depending on the type of microorganism to be obtained, Amies medium, Stuart medium, or Copan Universal Transport Medium® (UTM®) or even weak saline solutions based on table salt solutions or phosphate buffered saline solutions (PBS). According to particular embodiments of the disclosure, the transport medium can also comprise a nutrient medium, also known as culture medium, for the maintenance of certain micro-organisms.

In particular, the microfluidic apparatus can be a microfluidic cartridge for receiving and processing the sample, wherein the cartridge can be incorporated into an analytical device for actuating the processing of the sample in the cartridge.

The sample is incorporated into the microfluidic apparatus along with the cells and transport medium contained in the sample, in particular into a sample input chamber of the apparatus. The mixture of sample and transport medium, in particular the part of this mixture incorporated into the apparatus, is also further referred to below as “the sample” for short. Then, a lysis of cells in the sample occurs in order to release nucleic acids from the cells. This lysed sample, which in particular further comprises the transport medium, is subsequently applied to a solid phase of the apparatus in order to bind the released nucleic acids onto the filter. In the case of a filter as a solid phase, in particular a silica filter, the lysed sample can be conveyed, in particular pumped, through the filter. After binding of the nucleic acids to the filter, in particular after a specified duration has elapsed after application of the lysed sample to the solid phase or after the end of a pumping of the lysed sample through the filter, the filter is preferably washed in order to remove residues from the sample, in particular constituents, for example chaotropic salts, of the binding or lysing medium from the filter. The wash buffer used for this purpose is preferably positioned upstream of the apparatus. Subsequently, the nucleic acids bonded to the filter and now purified can be eluted with an elution medium, in particular an elution buffer, i.e. dissolved from the filter, in particular for a subsequent nucleic acid amplification.

The lysis of the cells can generally be performed (bio)chemically via mixing with a lysing medium comprising chemical or enzymatic lysis reagents, mechanically, in particular via ultrasonic exposure, and/or thermally via heating of the cells. Preferably, the lysing of the sample is carried out by adding a lysing medium to the sample, wherein the lysing medium is preferably stored in the microfluidic apparatus. The lysing medium is preferably an aqueous solution. Preferably, the lysing medium comprises chaotropic substances, in particular in a concentration from 1.5 to 4.5 mole (M, mole as a unit for mole per liter). The chaotropic substances can in particular be chaotropic salts, such as barium salts, guanidinium hydrochloride, thiocyanates such as guanidinium thiocyanate, perchlorates such as sodium perchlorate or sodium chloride. Preferably, the lysing medium, in particular the lysing buffer, does not comprise alcohol, in particular no ethanol and/or no propanol.

The binding of the nucleic acids released by the lysis in the sample is supported by a binding medium added to the sample. The binding medium, which is preferably positioned upstream of the apparatus and is preferably an aqueous solution, can be a binding buffer that is added to the sample. According to a special design, the lysing medium can form part of the binding medium or be identical to the binding medium. The binding medium and/or the lysing medium preferably have a concentration of at least 1.5 mole of chaotropic salts for a binding effect for binding the nucleic acids to the filter, preferably a concentration of between 1.5 and 4.5 mole, preferably between 3.0 and 3.5 mole. For example, the binding medium and/or the lysing medium comprise guanidinium thiocyanate and/or guananidine hydrochloride and/or the other chaotropic salts mentioned above as the chaotropic salt. Preferably, the binding medium, in particular the binding buffer, does not comprise alcohol, in particular no ethanol and/or no propanol. The lysis, preferably the addition of the lysing medium, and/or the addition of the bind medium are preferably carried out in the sample input chamber or in another chamber of the apparatus, preferably not in the chamber with the filter for binding the nucleic acids.

The disclosure thus advantageously allows nucleic acids located within intact cells in a sample to be released, bonded to a solid phase of a microfluidic apparatus, and thus separated from other constituents of the sample. Through such a direct binding of the nucleic acids to the solid phase, the washing can be carried out thoroughly so that only negligible residues from the sample, from the transport medium, and above all from the binding or lysing medium remain on the solid phase, which further advantageously makes it possible to duplicate in particular parts of these nucleic acids via a PCR or isothermal amplification practically without inhibiting substances. The disclosure thus allows a hard lysing medium and/or binding medium to be used with chaotropic reagents instead of the soft lysing buffer used so far, on detergent-based lysing buffer, even for samples with intact cells in stabilizing transport media. While in the cell-capture approach, only the intact cells can be retained and washed at the solid phase due to insufficient binding conditions for nucleic acids, and only then can lysis occur, due to the lysis performed prior to the contact with the solid phase, according to the disclosure, residues of the lysed cells, in particular cell fragments and cell debris, can also be removed via purification, resulting in a more thorough purification. Furthermore, the disclosure also allows a higher yield of nucleic acids by binding to the solid phase, because, in the cell capture approach, cells are partially damaged or opened up due to the processing of the sample, and thus nucleic acids are released, which are then not retained by the filter during washing. In addition, it is particularly advantageous that nucleic acids can also be isolated from microorganisms having smaller diameters, such as especially viruses or microorganisms, without a (robust) cell wall, for example mycoplasmas, from the sample, which cannot be well-definedly retained by a filter in the cell-capture approach.

Furthermore, it is of particular advantage that the method is also applicable for samples that are incorporated in non-stabilizing, inactivating, or even lysing transport media such as Copan eNAT™ or Roche cobas® PCR medium. Due to the lack of a cell stabilizing or even lysing effect, at least some of the cells in the sample are opened up and the nucleic acids located therein are released, which is however not critical for the further course of the method. Thus, the method can be used for a wide variety of usable transport media and effectively for all commonly used transport media for such samples. Thus, if a lysing transport medium, for example Copan eNAT™, is used for the sample transport, the method step of lysing the cells is carried out at least in part by mixing them with the transport medium and prior to the receipt of the sample in the microfluidic apparatus. In order to ensure a thorough lysis and to support the binding to the solid phase, a further step of lysing within the apparatus can additionally be carried out even after receipt, in particular by adding a lysing medium to the received sample.

To further support the binding of the nucleic acids to the filter, the binding medium and/or the lysing medium can contain substances for lowering pH, in particular buffer systems for setting a pH between 4.0 and 8.0, preferably between 5.0 and 7.0. If the mixture consisting of the sample, (preferably stabilizing) transport medium, and lysing and/or binding buffer, which comes into contact with the solid phase, in particular the filter, has a pH value less than 7, preferably between 5 and 7, the binding of the nucleic acids is in particular clearly supported with a silica filter.

According to a particularly advantageous further development of the disclosure, a processing medium is added to the sample. In the course of the disclosure, it has been discovered that by adding a processing medium, a chaotropic effect of the binding or lysing medium can be clearly supported, in particular in transport media containing substances having a cosmotropic effect. The cosmotropic effect existing in such a processing medium, in particular due to cosmotropic salts in the transport medium, can advantageously be reduced in a targeted manner. To this end, the processing medium preferably comprises a chelating agent containing ammonium ions that forms complexes with alkaline earth ions.

The processing medium can contain, as a chelating agent, at least one salt selected from the group consisting of ammonium hydrogen citrate, di-ammonium hydrogen citrate, ammonium citrate, ammonium chloride, ammonium acetate, tetramethyl ammonium citrate, and tetramethyl ammonium thioglycolate. The processing medium can comprise in particular a chelating agent disclosed in the publication DE 10 2020 203 035 A1. The content of the publication DE 10 2020 203 035 A1 with the filing date Mar. 10, 2020 and the disclosure date Sep. 19, 2021 is included as part of the disclosure. Such a chelating agent has the further advantage that by setting the chemical environment, the solubility product of particulate matter in the sample, in particular from a transport medium, such as Amies, can be displaced such that the particulate matter transitions into solution and passes through the solid phase, in particular the silica filter, during the filtration step and does not condense on it. The processing medium thus advantageously supports the use of the presented uniform and practically universal method for the purifying nucleic acids from samples in stabilizing and non-stabilizing transport media by conveying the use of a hard buffer from the BWE method and reducing or even eliminating disadvantages from the cell-capture method.

The processing medium, which is preferably positioned upstream of the cartridge, can be added to the sample before or after the addition of the lysing medium. By selecting the order and amount of the addition of the processing medium and the lysing medium, the dilution effect can also be controlled in a targeted manner by the addition. With regard to the effect of the chaotropic substances in particular, the addition of the processing medium can have two competing effects, which can be controlled in terms of their extent, in particular via the concentration of the above-mentioned chelating agents in the processing medium, namely on the one hand, a compensation of the effect of cosmotropic substances and on the other hand, a reduction of the chaotropic effect by dilution. According to the particular design, the processing medium forms a part of the binding medium or the lysing medium. For example, the processing medium can be mixed with the binding medium and/or the lysing medium before the resulting mixture is added to the sample. In a further embodiment, the lysing medium can first be added to the sample. Only after lysis has been performed, especially after a specified time has elapsed, is the mixture of sample and lysing medium or a part thereof mixed with the processing medium and the binding medium. This is particularly advantageous when lysis without incorporating the processing medium functions sufficiently or even better and the processing medium and the binding medium are aligned with one another, for example already present as a common medium, and preferably also aligned with the lysing medium, in order set advantageous binding conditions after mixing the three media.

The subject-matter of the disclosure is also a microfluidic apparatus, in particular configured for carrying out the method according to the disclosure, wherein a binding medium, a lysing medium, and/or a processing medium are positioned upstream in the apparatus such that they can be mixed with a sample input into a sample input chamber of the apparatus prior to applying the sample to a solid phase, in particular to a filter of the apparatus. As stated above, the apparatus, which can in particular be embodied as a microfluidic cartridge, can comprise a respective chamber for pre-storing the binding medium, lysing medium, and/or processing medium.

According to the preferred embodiment of the disclosure, the binding medium, the lysing medium, and/or the processing medium are positioned upstream as a common combined medium in the apparatus, in particular in the form of a buffer, for example in liquid form as reagent bars or in dried form, in a reagent chamber of the apparatus or cartridge. By pre-storing the binding medium and the processing medium, an effectively universal apparatus is created, in particular a cartridge, which is suitable for all commonly used transport media, whether lysing or cell-stabilizing, and which can provide a purification of nucleic acids from samples in these transport media.

For example, the apparatus comprises a combined medium, which, as already stated above, depicts and thus replaces both the functions of the lysing medium and the functions of the binding medium, or alternatively the functions of the binding medium and the processing medium. Preferably, the combined medium assumes the functions of the binding medium, the lysing medium, and the processing medium. For example, the combined medium is prepared by mixing lysing buffer, binding buffer, and processing buffer. Such combined medium further supports the universality of the apparatus with respect to the transport media described above.

The term “medium,” in particular in the case of lysing medium, binding medium, processing medium, or combined medium, can be understood to mean a solution, in particular an aqueous solution. A “buffer,” in particular in the case of the lysing buffer, binding buffer or processing buffer, can be understood in the narrower sense of the chemical term of buffer, i.e. in particular a mixture of substances, whose pH (concentration of the opanium ions) changes significantly less when an acid or base is added than would be the case in an unbuffered system. Alternatively, such a buffer can preferably also be more generally understood as a solution, in particular an aqueous solution.

### BRIEF DESCRIPTION OF THE DRAWINGS

Exemplary embodiments of the disclosure are shown schematically in the drawings and explained in more detail in the description hereinafter. Like reference signs are used for elements illustrated in the various drawings having a similar effect, wherein a repeated description of the elements has been omitted.

Shown are

FIG. 1 a flow chart of an exemplary embodiment of a method according to the disclosure, and

FIG. 2 an exemplary embodiment of the microfluidic apparatus according to the disclosure.

### DETAILED DESCRIPTION

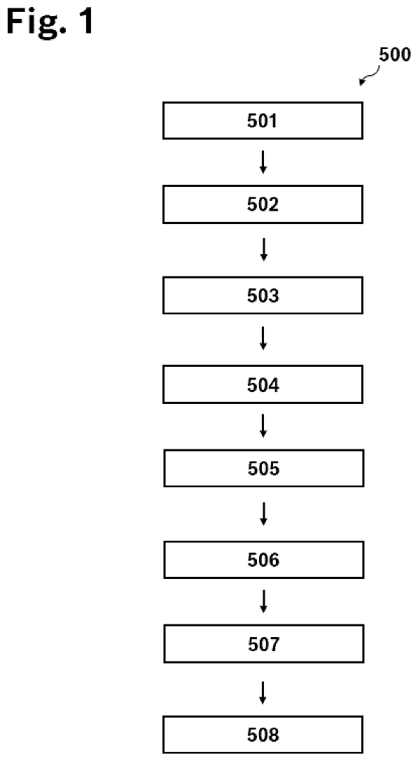

FIG. 1 shows a flowchart of an exemplary embodiment of the method 500 according to the disclosure.

In a first step 501, a biological sample is provided, which is to be tested for the presence of pathogens, namely using a PCR assay in a microfluidic point-of-care molecular diagnostic system. The biological sample, which is for example a swab sample, a patient's sputum, or patient's blood, is placed directly into a transport medium after receipt, for example, via a flocked swab into a vessel comprising the transport medium, for example, using the Copan eSwabs™ swab and transport system (Copan Liquid Amies Elution Swab), wherein the transport medium comprises a stabilizing medium for obtaining the cells of the potential pathogens in the sample. For example, depending on the type of microorganism to be detected, the transport medium is an Amies medium or UTM® or also a weak saline solution, for example 0.9% saline or PBS.

The mixture comprising the biological sample and the transport medium comprising the transport medium, hereinafter referred to jointly as “the sample,” are input into a microfluidic apparatus in a second step 502, for example, a few hundred microliters of this mixture, which is also referred to briefly as “the sample” in the following.

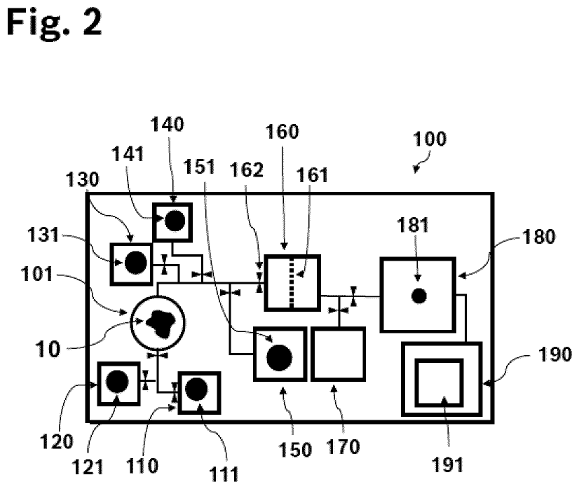

FIG. 2 schematically shows excerpts of a structure of a microfluidic cartridge 100. The cartridge 100 can comprise in particular a layer structure, having a fluid layer comprising a majority of the entire fluidic network of the cartridge and a pneumatic layer, as well as an extensible membrane layer arranged between the fluid layer and the pneumatic layer, which is extended in some places in chambers of the fluidic layer and the pneumatic layer by the pressures applied via a pneumatic interface, depending on the flow, in order to move fluids, including reagents, in the cartridge. The input of the sample 10 is made into a sealable sample input chamber 101.

In a third step 503, a lysing of the cells in the sample occurs by adding a lysing medium 111 in order to release the nucleic acids from the cells and obtain a lysed sample. The lysing medium 111 is stored in a first reagent chamber 110 that is fluidly connected to the sample input chamber 101, for example in the form of a reagent latch that can be opened via mechanical force, and can be conveyed into the sample input chamber 110 via, for example, a corresponding deflection in some places of the extensible membrane layer, and mixed with the sample 10 for the lysis of the cells.

In this example, the lysing medium 111 comprises a lysing buffer with chaotropic substances in a concentration of between 1.5 and 4.5 mole, for example 3.7 mole. The chaotropic substances can be chaotropic salts, for example guanidinium thiocyanate, for example as a 1:1 composition of thiocanic acid and guanidine, with a content of between 25 and 40 wt % in an aqueous solution.

Preferably, a processing medium 121 is previously added to the sample 10, for example as a processing buffer, which can be positioned upstream of a second reagent chamber 120 and contains at least one salt as a chelating agent selected from the group consisting of ammonium hydrogen citrate, di-ammonium hydrogen citrate, ammonium citrate, ammonium thioglycolate, ammonium chloride, ammonium acetrate, tetramethyl ammonium citrate, and tetramethyl ammonium thioglycolate. The processing medium 121 can at least partially reduce any cosmotropic effects introduced by the transport medium in the sample 10 and thus indirectly obtain the chaotropic effect of the lysing buffer 111 added to the sample. By aligning the concentrations of the constituents in the processing medium 121 and lysing buffer 111 in view of the composition of the nutrient medium used, an effective operating point for the lysis of the cells in the sample can thus be set. Alternatively, the processing medium 121 can be added to the sample only after the addition of the lysing medium 111. In particular in other designs, in particular depending on the desired operating point, the addition of the processing medium can be omitted entirely.

The mixing of the sample 10 with lysing buffer 111 and/or with the processing medium 121 can take place in the sample input chamber 101.

In a fourth step 504 of the method 500, a binding buffer 131 positioned upstream in a third reagent chamber 130 is added to the sample, for example, also via conveyance into the sample input chamber 101, in order to set suitable conditions for binding the nucleic acids released in the sample onto a filter. For example, the filter can be a silica filter 161 based on a porous silica membrane, for example POREX®, and can be in the form of a filter frit, which is located in a further chamber 160, called the filter chamber 160. For the setting of the binding conditions, the binding buffer 131 can comprise chaotropic substances, in particular chaotropic salts. For the setting of these binding conditions, the binding buffer 131 can be composed in alignment with the other buffers 111, 121 such that the mixture resulting from the mixing of the binding buffer 131 with the sample 10, preferably the lysing buffer 110 and/or the processing medium 121, has a concentration of the chaotropic salts in a range between 1.6 to 2.2 mole. The addition of the processing medium 121 as described above can thereby lower the concentration of the chaotropic substances or salts, for example from 3.7 mole to 1.5 M, in order to cause an adequate but not too strong binding of the nucleic acids to the filter 161 while reducing the influence of the chaotropic substances for the subsequent steps. In order to support the binding conditions, substances can be added to the lysing, binding, or processing medium in order to lower the pH, for example to give the mixture contacting the solid phase 161 comprising the sample a pH between 5 and 7. For example, these substances can be a citric acid/citrate buffer and/or an acetic acid/acetate buffer and/or a thioglycolic acid/thioglycolate buffer. These buffers are in particular suitable for setting a pH in the range of 5 to 6. If a citric acid/citrate buffer is to be used, the chelating agent is preferably an ammonium hydrogen citrate, ammonium citrate, or tetramethyl ammonium citrate as the processing medium, and citric acid is additionally added thereto. If the buffer is intended to be an acetic acid/acetate buffer, the chelating agent is preferably ammonium acetate, and acetic acid is added thereto. If a thioglycolic acid/thioglycolate buffer is to be used, the chelating agent is preferably ammonium thioglycolate or tetramethyl ammonium thioglycolate, and thioglycolic acid, also referred to as mercaptoacetic acid, is further added. In principle, it is also possible for the chelating agent to contain only one of the salts suitable for buffer formation, i.e. a hydrogen citrate, a citrate, an acetate, or a thioglycolate, and the corresponding acid is then released from it by the addition of another organic acid. Mandelic acid is in particular suitable for this purpose. The addition of a further organic acid, for example mandelic acid, to an already existing system of salt and corresponding acid can also be provided.

The binding buffer 131, as also described above, can in particular be a combined binding and lysing buffer, i.e. a buffer that both lyses the cells in the sample and creates suitable chemical conditions for binding the released nucleic acids to the filter 161. For example, such a combined buffer, which is preferably also positioned upstream of the cartridge in one of the reagent chambers 110, 130, has between 2.5 and 3.5 mole guanidinium thiocyanate and a pH between 6-7. In particular, due to space requirements, instead of the individual media, a common medium or combined medium, in particular a common buffer, can also be used and positioned preferably upstream of the cartridge in one of the reagent chambers 110, 120, 130, wherein the common medium provides the functions of lysing medium, binding medium, and processing medium. To this end, the common medium can be prepared, for example by mixing lysing buffer, binding buffer, and processing buffer, either outside the cartridge or inside the cartridge, for example not until performing the method by mixing in one of the reagent chambers 110, 120, 130.

After a specified time, for example a few seconds to a few minutes, the sample set to the aforementioned binding conditions is conveyed onto the filter 161 in the filter chamber 160 that is fluidly connected to the sample input chamber 101 in a fifth step 505. Preferably, this fluidic connection is separable by a valve 162. For example, the application onto the filter 161 can occur by pumping the sample 10 via the filter 161 into a waste chamber 170 of the cartridge 100.

In a sixth step 506 of the method 500, the filter 161 is washed with the nucleic acids bonded thereto with a wash buffer 141 that is positioned upstream of the cartridge 100 in a fourth reagent chamber 140 in order to remove residues from the sample, in particular constituents, for example chaotropic salts, of the binding or lysing medium, from the filter. The wash buffer can be a buffer or solution commonly used for the purifying nucleic acids, but preferably without ethanol, wherein the wash buffer preferably contains less than 0.1 mole per liter of chaotropic substances, in particular chaotropic salts. For example, the wash buffer comprises a polyethylene glycol/water mixture.

In a seventh step 507 of the method 500, an elution buffer 151 is used as the elution medium in order to lift the binding conditions and thus detach the nucleic acids from the filter 161. For this purpose, the elution buffer 151 can be stored upstream in a fifth reagent chamber 150 fluidically connected to the filter chamber 160. The elution buffer can be a typical buffer for dissolving nucleic acids from silica, for example distilled or buffered water, possibly with surface-active substances such as polysorbate 80 (Tween® 80).

In a preferably eighth step 508, a further processing of the eluted nucleic acids can take place, for example an amplification of portions of the nucleic acids with the aid of a (quantitative real-time) polymerase chain reaction, an isothermal amplification, or a ligase chain reaction in a further chamber 180 (analysis chamber 180 for short) for detection of the nucleic acids and thus associated pathogens in the sample. For example, in the analysis chamber 180, there can be a PCR master mix in the form of a PCR bead 181. A direct mixing of the elution buffer 151 with the nucleic acids therein is unproblematic, because there are no longer any, or negligibly few, chaotropic substances that could impair the amplification.

The cartridge 100 can comprise a further chamber as a detection chamber 190, in which, for example, a micro array 191 with immobilized probes can be arranged for hybridizing the nucleic acid portions sought and duplicated, which can then be optically read out via, for example, fluorescence spectroscopy.

As noted above, this method 500, and in particular the microfluidic apparatus 100 described in FIG. 2, can also be used for samples received in non-stabilizing, inactivating or even lysing transport media such as Copan eNAT™ or Roche cobas® PCR Medium.

What is claimed is:

1. A method for purifying nucleic acids from cells in a sample with transport medium for nucleic acid amplification, with a microfluidic apparatus, comprising:
receiving a sample into the microfluidic apparatus, wherein the sample comprises cells having nucleic acids contained therein, and wherein the sample comprises a stabilizing transport medium for the cells;
lysing the cells in the sample by adding a lysing medium in order to release the nucleic acids from the cells and obtain a lysed sample;
applying the lysed sample onto a solid phase of the apparatus, wherein the solid phase of the apparatus is a filter, and wherein the lysed sample comprises a binding medium for binding the released nucleic acids onto the filter;
washing the filter in order to remove residues from the sample, wherein the residues include constituents, and wherein the constituents include chaotropic salts of the binding or lysing medium; and
eluting the nucleic acids bonded to the filter with an elution medium.

2. The method according to claim 1, wherein the lysing of the sample comprises an addition of a lysing medium to the sample, and wherein the lysing medium is positioned upstream of the microfluidic apparatus.

3. The method according to claim 2, wherein the lysing medium forms at least a portion of the binding medium.

4. The method according to claim 1, wherein:
for a binding effect for binding the nucleic acids to the filter, the binding medium and/or the lysing medium have a concentration of at least 1.5 mole of chaotropic salts.

5. The method according to claim 1, wherein:
for a binding effect for binding the nucleic acids to the filter, the binding medium and/or the lysing medium comprises substances for lowering the pH value upon mixing with the sample.

6. The method according to claim 1, wherein the lysing medium and/or the binding medium comprises guanidinium thiocyanate or guanidine hydrochloride as the chaotropic salt.

7. The method according to claim 1, wherein a processing medium is added to the sample in order to support a chaotropic effect of the binding medium and/or lysing medium.

8. The method according to claim 7, wherein the processing medium forms part of the binding medium or lysing medium.

9. The method according to claim 7, wherein the processing medium contains, as a chelating agent, at least one salt selected from the group consisting of ammonium hydrogen citrate, di-ammonium hydrogen citrate, ammonium citrate, ammonium chloride, ammonium acetate, tetramethyl ammonium citrate, and tetramethyl ammonium thioglycolate.

10. The method according to claim 1, wherein the lysing of the cells takes place in a chamber other than the chamber comprising the filter.

11. The method according to claim 1, wherein the sample comprises an inactivating, lysing, transport medium for the cells, and wherein the lysing of the cells in the sample occurs at least in part prior to the receipt of the sample into the microfluidic apparatus through the transport medium.

12. A microfluidic apparatus configured to carry out the method according to claim 1, wherein a binding medium, a lysing medium, and/or a processing medium are positioned upstream in the apparatus in such a way that they can be mixed with a sample input into a sample input chamber of the apparatus prior to applying the sample onto a solid phase of the apparatus.

13. The microfluidic apparatus according to claim 12, wherein the binding medium, the lysing medium, and/or the processing medium are positioned upstream of the apparatus as a common combined medium.

14. The method according to claim 1, wherein:
for a binding effect for binding the nucleic acids to the filter, the binding medium and/or the lysing medium have a concentration between 1.5 and 4.5 mole of chaotropic salts.

15. The method according to claim 1, wherein the addition of the lysing medium and/or the addition of the binding medium takes place in a chamber other than the chamber comprising the filter.

16. The method according to claim 1, wherein the addition of the lysing medium and/or the addition of the binding medium takes place in a sample input chamber of the microfluidic apparatus.

17. The method according to claim 1, wherein the lysing of the cells takes place in a sample input chamber of the microfluidic apparatus.

18. A microfluidic apparatus configured to carry out the method according to claim 1, wherein a binding medium, a lysing medium, and/or a processing medium are positioned upstream in the apparatus in such a way that they can be mixed with a sample input into a sample input chamber of the apparatus prior to applying the sample onto a filter of the apparatus.
